(19) United States
(12) Patent Application Publication
(10) Pub. No.: US 2010/0222861 A1
(43) Pub. Date: Sep. 2, 2010

(54) System for Treating Lesions on a Blood Vessel Bifurcation

(76) Inventor: Alain Dibie, Paris (FR)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 12/223,845

(22) PCT Filed: Feb. 8, 2007
(86) PCT No.: PCT/EP2007/051205

§ 371 Date: May 20, 2010

(30) Foreign Application Priority Data
Feb. 9, 2006 (FR) 0601183

(51) Int. Cl. A61F 2/84; A61F 2/82

(52) U.S. Cl. 623/1.11; 623/1.15

(57) ABSTRACT

The invention relates to a system for treating lesions on a blood vessel bifurcation presenting a first branch (B1) and a second branch (B2), said system comprising: a first endoprosthesis (100) designed to be positioned in the first branch; a second endoprosthesis (200) designed to be placed in the second branch (B2) by engagement through an orifice (140) of the first endoprosthesis (100) once said orifice (140) has been dilated; and an insertion instrument (400) which has a longitudinal axis (1) and which comprises: a first balloon (500) designed to be introduced into the second branch through the orifice (140) of the first endoprosthesis (100) and to dilate said orifice (140); and a second balloon (600) which is aligned with the first balloon (500) along the longitudinal axis (1) and which is designed to receive the second endoprosthesis (200) and dilate it.

The present invention relates to the field of systems for treating lesions on blood vessel bifurcations.

More precisely, the present invention relates to a system for the fitting of stents for this purpose.

One is conventionally familiar with the treatment of stenosis or narrowing found in coronary arteries by the insertion of stents formed of perforated tubular bodies in a mesh that is expandable, using a guide, at the site of the stenosis.

Most often, the expansion of these stents is achieved by the inflation of a balloon located inside the stents, the balloon then being withdrawn.

This usual system for the insertion of a stent has already rendered considerable service. However, it is not totally satisfactory.

In particular, the applicant has observed that it is not totally satisfactory when, as is frequently the case, the stenosis is located at a blood vessel bifurcation.

In fact, in this case, the arrangement of two separate stents intended to be respectively in the different branches of the bifurcation and whose relative positioning must be adjusted as accurately as possible in order to cover at least the bifurcation zone, requires the use of two separate insertion systems in two procedures that are made in parallel.

These two separate procedures increase the risks of crossing the guides used to insert the stents at the bifurcation zone, thus complicating the treatment of the stenosis sites at blood vessel bifurcations.

A first objective of the present invention is to perfect the systems for insertion the stents in order to facilitate and improve the treatment of stenosis at blood vessel bifurcations.

A second aim of the present invention is to propose a system for the fitting of stents that leaves to the operator the choice of insertion either one or two stents at the bifurcation zone of a blood vessel during the same procedure.

Another aim of the invention is to propose an insertion system that limits the risks of crossing the guides between two insertion instruments associated with two stents, each intended for one branch of a vessel bifurcation.

It is also desirable to propose an insertion system for stents that is simple to use and to manufacture while still preserving maximum effectiveness.

These objectives are reached in the context of the present invention by virtue of a system for treating lesions on a blood vessel bifurcation that includes a first branch and a second branch, the system being, characterised in that it includes:
a first stent adapted to be positioned, in part, in the first branch of the bifurcation;
a second stent adapted to be placed in the second branch of the bifurcation and to be inserted into this second branch by passing through an orifice of the first stent once the latter has been dilated and,
an insertion instrument with a first longitudinal axis, said insertion instrument including:
a first balloon adapted to be inserted into the second branch through the orifice of the first stent and to dilate this orifice;
a second balloon, aligned with the first balloon along the first longitudinal axis, adapted to receive the second stent and to dilate it.

According to another aspect, the invention proposes a system for treating lesions on a blood vessel bifurcation, which includes:
a first insertion instrument associated with a main stent intended to be placed, in part, in a first branch of the bifurcation, with a first longitudinal axis, and that includes at least one first guiding channel lying along the said first axis, designed to receive a first guiding element of the first insertion instrument;
a second insertion instrument associated with a secondary stent intended to be placed, where appropriate, in a second branch of the bifurcation, with a second longitudinal axis, and that includes at least one second guiding channel lying along the said second axis designed to receive a second guiding element, the said second guiding element being intended to direct the second insertion instrument into the second branch of the bifurcation,

the said system being characterised in that the second insertion instrument is in possession of reception means adapted to receive, in part, the first guiding element, and the first insertion instrument is in possession of means for reversible attachment of the said first guiding element to the reception means so as to hold the two insertion instruments together during their progression on the second guiding element up to the bifurcation.

The invention also relates to a method for treating lesions on a blood vessel bifurcation, which includes a first branch and a second branch, and where the method includes steps:
to position a first stent, in part, in the first branch of the bifurcation; and
to position a second stent in the second branch of the bifurcation, the second stent being inserted into this second branch by passing through an orifice of the first stent once the latter has been dilated;

in which the step for positioning of the second stent is made by means of an insertion instrument with a first longitudinal axis, and that includes a first balloon and a second balloon aligned with the first balloon along the first longitudinal axis, and that includes substeps to:
insert the first balloon into the second branch through the orifice of the first stent and to dilate the said orifice;
receive the second stent on the second balloon and to dilate the said second stent.

The invention will be understood better and other advantages and characteristics will appear more clearly on reading the description that follows and which is given by way of a non-limiting example with reference to the appended drawings, in which:

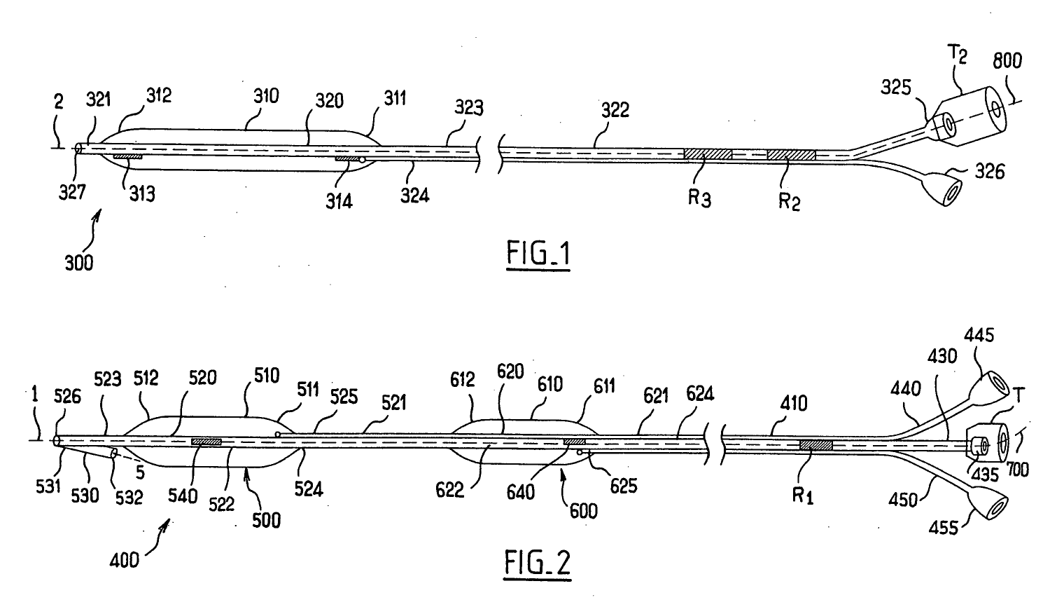

FIG. 1 illustrates a schematic side view of an insertion instrument associated with a main stent devoted to a first branch of a bifurcation in a blood vessel;

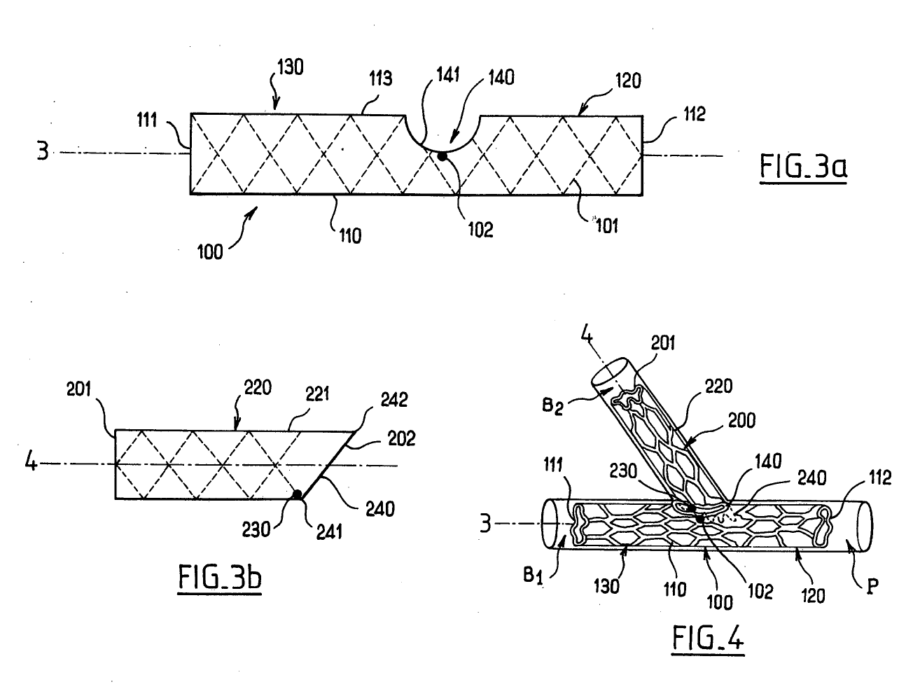

FIG. 2 illustrates a schematic side view of a double-balloon insertion instrument, associated with a secondary stent devoted to a second branch of a bifurcation in a blood vessel;

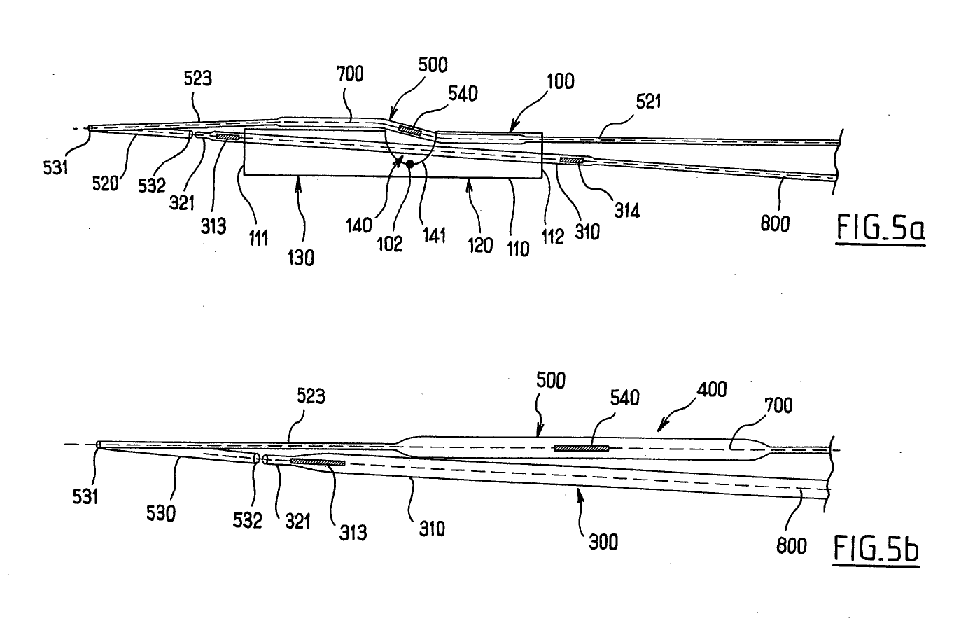

FIGS. 3a and 3b illustrate cross-section views, respectively, of a main stent devoted to a first branch of a bifurcation in a blood vessel and a secondary bevelled stent devoted to a second branch of the bifurcation;

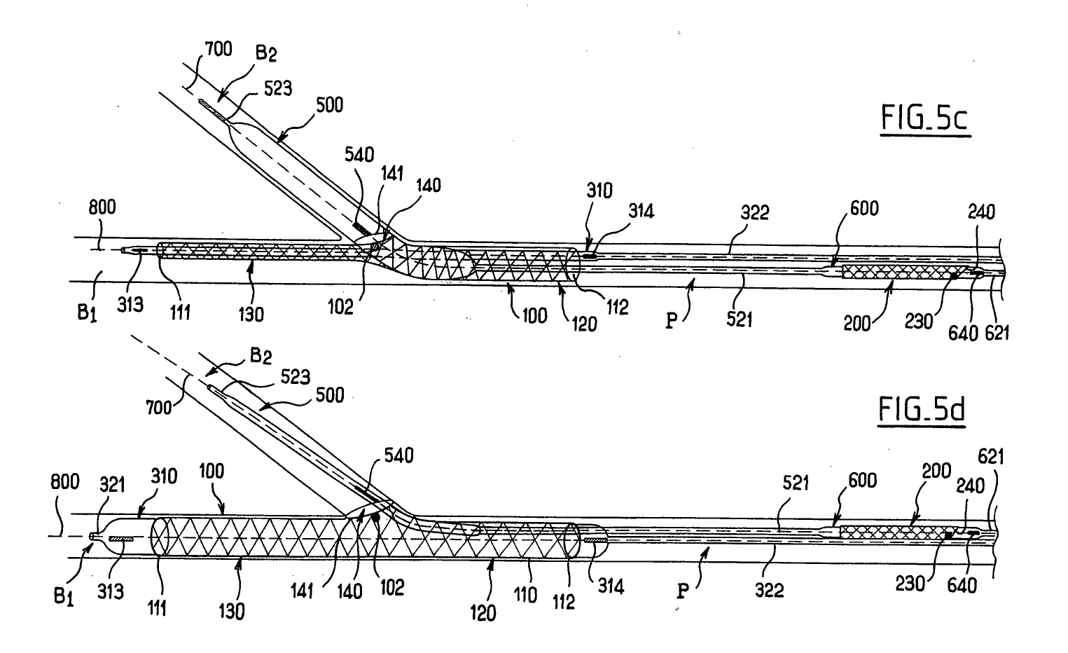

FIG. 4 illustrates a view in perspective of the stents of FIGS. 3a and 3b positioned at the bifurcation in a blood vessel;

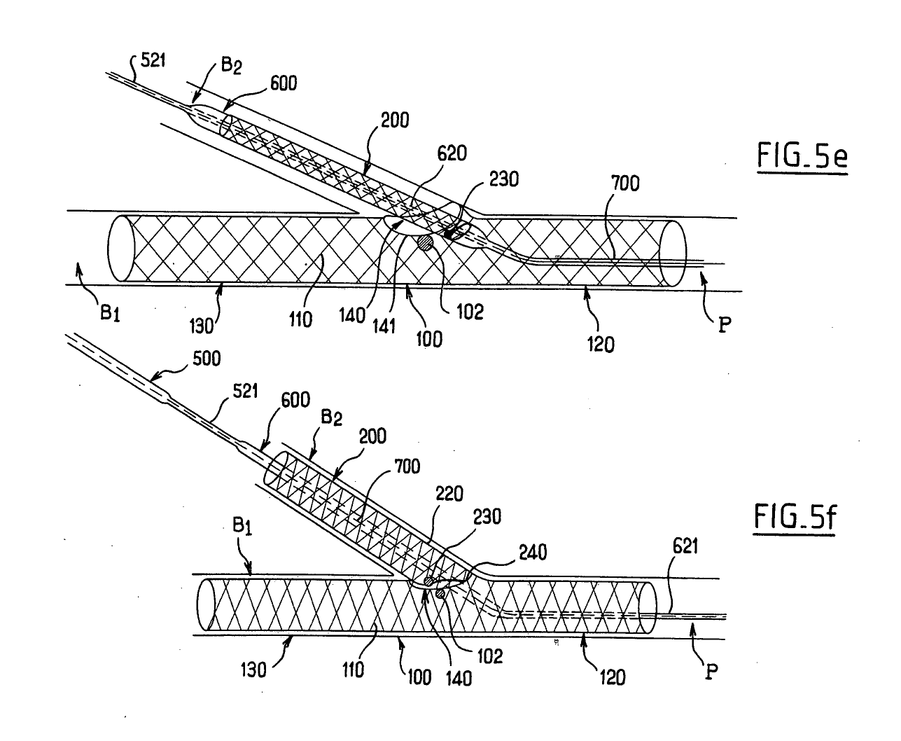

FIG. 5a illustrates a side view of a system for the fitting of stents according to the invention, that includes the double-balloon insertion instrument of FIG. 2 fitting onto the insertion instrument of FIG. 1 in a stent;

FIG. 5b illustrates a side view of the insertion of a system for the fitting of stents according to the invention, that includes the double-balloon insertion instrument of FIG. 2 fitting onto the insertion instrument of FIG. 1;

FIG. 5c illustrates a side view of the deployment of the stent carried by the insertion instrument of FIG. 1, by inflation of a first balloon of the double-balloon insertion instrument of FIG. 2;

FIG. 5d illustrates a side view of the deployment of the stent carried by the insertion instrument of FIG. 1, by deployment of the balloon of the latter;

FIG. 5e illustrates a side view of the insertion of the secondary stent by the associated double-balloon insertion instrument in a branch of the bifurcation in a blood vessel;

FIG. 5f illustrates a variant of FIG. 5c, with the secondary stent deployed;

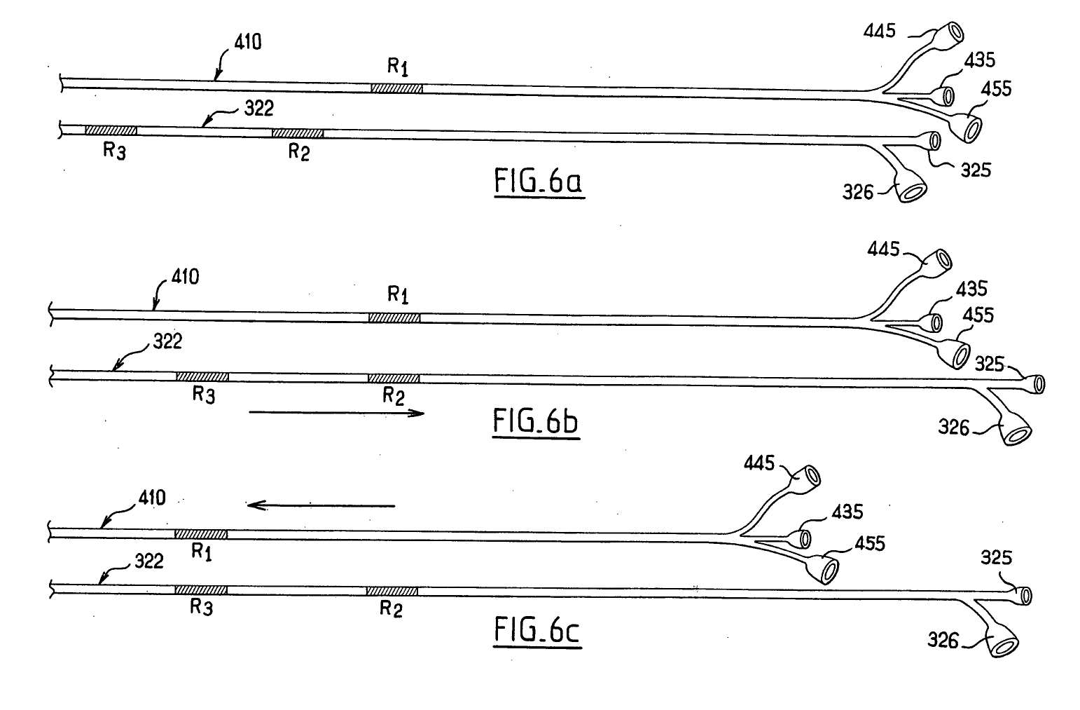

FIGS. 6a, 6b, and 6c illustrate the coordinated relative movements of the two insertion instruments of an insertion system according to the invention during insertion of stents at the bifurcation in a blood vessel.

### 1. A System for the Fitting of Stents for Treating Lesions on Blood Vessel Bifurcations

We will first describe a system for the fitting of stents for treating lesions on blood vessel bifurcations according to the present invention.

a. The Instruments of the Insertion System

This insertion system will be used to insert either one or two stents, according to the needs of the blood vessel (V) to be treated, during a given procedure.

It includes, firstly, a first insertion instrument 300 with a main balloon 310 designed to dilate a so-called main stent 100 intended to be placed in a first branch B1 of the bifurcation, and a main section P, upstream of the bifurcation in a blood vessel V to be treated, as can be seen in particular in FIG. 4.

This main balloon 310 is associated with a guiding channel 323 that is capable of receiving a first guiding element 800 intended to direct the balloon into the first branch B1 of the bifurcation.

Secondly, the insertion system includes a second double-balloon insertion instrument 400 dedicated to the insertion, where appropriate, of a so-called secondary stent 200 in the second branch B2 of the bifurcation in the blood vessel V to be treated.

This second insertion instrument 400 is designed to mate with the first.

It includes a first balloon 500 adapted to be inserted into the second branch B2 through an orifice 140 of the main stent 100, the first balloon 500 being also adapted to dilate the orifice 140 of the main stent 100.

It also includes a second balloon 600, adapted to receive the secondary stent 200 and to dilate it, the said stent being intended to be inserted into the second branch B2 by insertion through the orifice 140 of the first stent 100, once the latter has been dilated.

Secondly, it advantageously includes reception means 530 that are capable of receiving, in part, the first guiding element 800 of the first insertion instrument 300, so as to hold the two insertion instruments 300 and 400 together during the progression of the insertion system toward the bifurcation in the blood vessel V to be treated.

We will now describe, more precisely, the structure of the second double-balloon insertion instrument 400, represented in FIG. 2.

Essentially, this insertion instrument 400, with a longitudinal axis 1, includes the first 500 and the second 600 balloons, the more proximal, positioned in line along this axis 1.

These two balloons 500 and 600 are formed, respectively and preferably, of long tubular elements 510, 610, generally cylindrical, centred on the axis 1 and, whose respective ends 511, 512 and 611, 612 are generally rounded.

More precisely, these balloons 500 and 600 are respectively fixed onto two internal hollow tubes 520 and 620 lying parallel to the axis 1 and passing axially through the said balloons 500 and 600. They respectively include at least one guiding channel 522, 622 receiving a guiding element of the angioplasty flexible metal guide type 700.

The balloons 500 and 600 are extended, respectively, at their proximal ends 511 and 611 by two respective inflation tubes 521 and 621 lying substantially along the axis 1 and adapted to inflate the balloons.

These inflation tubes 521 and 621 preferably surround the guide 700. More precisely and preferably, each of these includes two channels: a first channel 524, 624 that receives the metal guide 700 and emerges into the guiding channel of the associated inner tube 520 and 620 and a second channel 525, 625 used for the inflation that emerges into the internal volume of the balloons 500 and 600.

It should be noted that the internal tubes 520 and 620 and aforesaid first channels 524 and 624 are insulated from the internal volume of the balloons 500 and 600.

Secondly, the first and second balloons 500 and 600, aligned along the axis 1, are separated by the inflation tube 521 from the first balloon 500, over a length of a few millimetres.

In addition, this inflation tube 521 is extended and opens into the internal tube 620 associated with the second balloon 600, which itself is extended into the inflation tube 621 of the latter.

Thus, the inflation tube 621 of the most proximal balloon 600 is composed, more precisely, of three channels, namely a guiding channel 624 that is common to the two balloons 500 and 600, which receives the metal guide 700, a first inflation channel 525 of the first balloon 500 that opens into the associated internal tube 520 and a second channel 625 used for inflation of the second balloon 600 that opens into the internal volume of this balloon 600.

As a consequence, the two inflation tubes 521 and 621 associated with the two balloons 500 and 600 are fixed together to the proximal end 611 of the second balloon 600 over a large part of their length.

They are preferably welded together.

They then form a common element 410 with three respective channels 430, 440, 450 respectively linked to the tubes and more precisely the aforementioned guide 624 and inflation 525, 625 channels of the latter.

Thus, the two balloons 500 and 600 are attached, joined over the major part of their inflation tubes 521 and 621 and they have a common guiding channel 624 receiving the metal guide 700 directing the assembly into the second branch B2 of the bifurcation.

As a consequence, they will move together in the course of a procedure, where required, for insertion the secondary stent 200.

In addition, the common element 410 of the second insertion instrument 400 is equipped at its proximal end, opposite to the second balloon 600, with two connection systems 445 and 455 with fluid sources and respectively linked to the inflation channels 440 and 450 allowing expansion of the balloons 500 and 600.

It also has means for reversible attachment and, more particularly, a connection system 435 with a torquer T and in association with the guiding channel 430 allowing the attachment of the guide 700.

For example, these connection systems 430, 440 and 450 can be of the type bearing the title “luer lock”.

In a variant, the connection means 445 and 455 for the inflation of the aforementioned balloons can be adapted to receive a conventional inflation syringe end-piece.

It is important that the aforementioned connection means 445 and 455 should allow separate expansion of the two balloons 500 and 600.

In its proximal part close to the connection systems, the common element 410 has a colour marker R1 that will be used to place the secondary stent 600 in the right position in the second branch B2 of the bifurcation.

This marker R1, with a length of the order of 20 mm, is positioned so as to correspond, during the movements of the second insertion instrument 400, to the markers located on the first insertion instrument 300, as will be described later in relation to FIG. 6.

In addition, each of the two balloons 500 and 600 is preferably equipped with radio-opaque markers. More precisely, the first balloon 500 has a marker 540 carried by its internal tube 520 and positioned at mid-length of the latter while the second balloon 600 has a marker 640 located at the proximal end 611 of the balloon 600 and carried by the associated internal tube 620.

In addition the internal tube 520 of the first balloon 500 is advantageously extended outside of the latter, at its distal end 512, opposite to the inflation tube 521, by a so-called nose tube 523 of the balloon 500 lying along the axis 1.

The guiding channel 522 associated with the internal tube 520 opens into the nose 523 of the first balloon 500 and the guide 700 emerges from the nose 523 by its free and open distal end 526.

This nose 523 of the first balloon 500 also has, at this end 526 of the reception means adapted to receive the first guiding element 800 guiding the first insertion instrument 300.

The two insertion instruments 300 and 400 of the stents 100 and 200 will then be able to fit together during the respective positioning of the latter in the branches B1 and B2 of the bifurcation as will be described later in relation to FIG. 5.

These reception means include a so-called auxiliary hollow tube 530, centred on an axis 5.

It lies along the axis 5 of the distal end 526 of the nose 523 of the first balloon 500 toward the first balloon 500 in a slightly inclined direction in relation to the axis 1.

It has a distal end 531 with a bevel, the bevel being determined by a plane parallel to the axis 1 and perpendicular to the plane of the sheet.

From this distal end 531, it is attached, by adhesion or welding, to the free end 523 of the nose of the first balloon 500 over the length of the bevel. This length is of the order of a few millimetres.

Secondly, this auxiliary tube 530 is adapted to receive, at its open proximal end 532, one end of the mandrel 800 or of the metal guide guiding the first insertion instrument 300.

Concerning this first insertion instrument 300, illustrated in FIG. 1, it includes a main balloon 310 centred on an axis 2 associated with an internal tube 320, an inflation tube 322 and a nose 321 that is substantially the same as those described in relation to FIG. 2 for the first balloon 500.

Thus, the first insertion instrument 300 includes a guiding channel 323 that is common to the different tubes 320, 322 and to the nose 321, adapted to receive the mandrel 800 guiding the insertion instrument 300 during its progression in the coronary artery toward the first branch B1 of the bifurcation.

This mandrel 800 opens out of the insertion instrument 300, at the free end 327 of the nose 321 of the balloon 310 and takes up a position in the auxiliary tube 530 of the second insertion instrument 400.

In order to allow the attachment of the mandrel 800 in the auxiliary tube 530, the inflation tube 322 of the main balloon 310 is equipped, at its proximal end, with means for reversible attachment, and more precisely with a connection system 325 with a torquer T2, and in association with the guiding channel 323.

The respective insertion instruments 300 and 400 of the two main 100 and secondary 200 stents are then attached, fixed together at their respective noses 321, 523.

In addition, the first insertion instrument 300 also includes a connection system 326 with fluid sources of the luer-lock type in association with an inflation channel 324 allowing the expansion of the main balloon 310.

In addition, on its inflation tube 322, it includes two colour markers R3 and R2 separated by a few tens of millimetres serving as markers during the placement of the secondary stent 200 in the second branch B2. The most distal marker R3, and the R2 marker each extends over a length of the order of 20 mm and are separated by a distance of about 27 mm for example.

The main balloon 310 also has two radio-opaque markers 313 and 314 carried by its internal tube 320 and positioned, respectively, at each of its two ends 312 and 311. These markers 313 and 314 extend over a length of the order of 2 mm.

According a particular embodiment of the invention, which is not limiting of course, the two respective insertion instruments 300 and 400 of the main 100 and secondary 200 stents have the following dimensions:
The first and second balloons 500 and 600 have a length that is adapted to take position in the second branch B2 of the bifurcation in the blood vessel V while the main balloon 310 has a length adapted to take position in the first branch B1 and in the main section P of the blood vessel V;
The first and second balloons 500 and 600 have a respective length of 14 mm and 12 mm and have a diameter in the inflated state of the order of 2.0 mm to 3.0 mm;
The main balloon 310 has a length of the order of 20 mm and an outside diameter in the inflated state of the order of 2.5 to 4 mm;
The external dimension of the attached insertion instruments 400 and 300 in the deflated state of the balloons 500 and 600 is preferably adapted to move along a guide catheter 6F;

The guiding channels 624, 524 and 323 of the two insertion instruments 400 and 300 have a length of the order of 110 to 135 cm and an inside diameter adapted to receive a flexible guide or metal mandrel with a diameter of 0.014 inches;
The first balloon 500 is separated from the second 600 by its inflation tube 521 by a distance of 30 to 34 mm;
The first balloon 500 has a nose with a length of 10.5 to 11 mm, while the nose 321 of the main balloon 310 extends over a length of the order of 3 mm;
The auxiliary tube 530 is attached to the nose 522 of the first balloon 500 over a length of the order of 6 mm.
   b. The Stents

We will now describe the two stents intended to be placed at the bifurcation zone, as can be seen in particular in FIG. 4.

Concerning the secondary stent 200, illustrated in FIGS. 3b and 4, and intended, where appropriate, to be positioned in the second branch B2 of the bifurcation in the blood vessel V to be treated, this includes a tubular section 220 centred on an axis 4.

The section 220 is preferably formed from a grill-type perforated tubular structure 221 so that the structure of the section 220 allows the expansion, in a straight section, of the latter.

The mesh-type pattern of the section can be the subject of many variants. It can, for example, be a pattern in the diamond or hexagonal form, as represented respectively in FIGS. 3b and 4 giving the appearance of a mesh during their expansion by the second internal balloon 600, on the internal wall of branch B2 of the bifurcation.

Since the basic structure of such expandable tubular elements 221 and the material constituting the latter are already familiar to those skilled in the art, these arrangements will not be described in detail in what follows.

As illustrated in FIG. 3b, the proximal end 202 of the section 220 intended to be placed at the origin of the second branch B2 of the bifurcation, at the sheath of the bifurcation, is fitted on its periphery with a bevel 240.

This bevel 240 can be determined by a plane that is inclined in relation to axis 4, and perpendicular to the plane of the sheet. Thus, the angular opening in the wall of the section increases from the proximal end 202 until it covers 360°, that is with a complete tubular form around axis 4 at the opposite end 201.

According a particular embodiment of the invention, given by way of a non-limiting example, the length of the bevel 240 is of the order of 7 to 9 mm, and the inside diameter of the secondary stent 200 is of the order of 20 to 3.0 mm.

An alternative embodiment specifies a secondary stent 200 that includes several bevels at its proximal end 202.

Secondly, to this end 202, the stent 200 has a radio-opaque marker 230. The latter is positioned at the end 241 of the bevel 240, the end closest to the distal end 201 of the section 220 and intended to be placed close to the sheath of the bifurcation.

In the developed position, as illustrated in FIG. 4, when the stent 200 is put in place in the second branch B2, the bevelled proximal end 202 positions itself harmoniously at the orifice 140 of the main stent 100 located at the entrance of the second branch B2, in order to cover the bifurcation zone as completely as possible.

In relation to the main stent 100, illustrated in FIGS. 3a and 4, this includes a tubular section 110 lying along axis 3 and centred on the latter.

This section 110 is formed of two coaxial distal 130 and proximal 120 portions, intended to be inserted respectively into the first branch B1 and the main section P of the blood vessel V.

At a longitudinal end 113 parallel to axis 3, upstream of the distal portion, the section 110 has the orifice 140 which is designed so that it takes the form of the entrance of the second branch B2 at which it will take position.

It appears, for example, in the form of a semicircle 141 opening toward the exterior of the section 110.

It is advantageously formed from a particular mesh.

This mesh is more open than the mesh that forms the grid-type perforated tubular element 101 forming the section 110.

Since such an element 101 has already been described with reference to FIG. 3b for the secondary stent 200, it will not be described in detail in what follows.

In the developed position, when the main stent 100 is put in place at the bifurcation in the blood vessel V, the orifice 140 takes up a position facing the second branch B2, ready to receive the second insertion instrument 400.

More precisely, the particular mesh or size of the orifice 140 is designed so that it can receive the first balloon 500 of the second instrument 400 and open wide without deforming during the expansion of this first balloon 500 at the entrance of the second branch B2 of the bifurcation.

Secondly, it is designed so that it will fit onto the shape of the secondary stent 200 crimped onto the second balloon 600 of the insertion instrument 400, the said stent 200 having to be inserted into the said open mesh in front of the entrance of the second branch B2 of the bifurcation and pass through it before being positioned in the branch B2, as will be described in relation to FIG. 5.

In addition, a radio-opaque marker 102, with a length of about 1 mm, is attached on the orifice 140. It can be place on the periphery of the latter. For example, in FIG. 3a, it is located at the centre of the circular arc 141.

In general, this radio-opaque marker 102 is positioned so as to correspond, during the installation of two stents 100 and 200 in the two branches of the bifurcation, to the radio-opaque marker 230 placed on the bevel 240 of the secondary stent 200.

In a non-limiting example of the invention, the section 110 of the main stent 100 has a length of the order of 17 to 18 mm, and the orifice 140 has an inside diameter of the order of 3 mm.

The main stent 100 is preferably a stent of the chromium-cobalt type.

### 2. Embodiment of the System for Fitting Stents for Treating Lesions on Blood Vessel Bifurcations

We will now describe an embodiment of the insertion system according to the invention and, more particularly, the insertion of one or two stents by this system.

a. Insertion of the System for the Fitting of Stents

To begin with, we place the insertion instrument 400 of the secondary stent 200 in the main stent 100 carried by the main balloon 310.

More precisely, with reference to FIG. 5a, the nose 523 of the first balloon 500 of the second insertion instrument 400 is inserted at the proximal end 112 of the proximal portion 120 of the main stent 100 and is then inserted outside of the main stent 100, through the mesh of the orifice 140, up to half the length of the internal tube 520 of the first balloon 500.

We then position the mandrel 800, by its distal end, in the guiding channel 323 of the first insertion instrument 300 until it emerges from the nose 321 of the main balloon 310.

As illustrated in FIGS. 5a and 5b, this mandrel 800 is then inserted into the auxiliary tube 530 of the second insertion instrument 400, the assembly being fixed by screwing the torquer T2 to the connection system 325 associated with the guiding channel 323.

The two noses 523 and 321 of the main balloon 310 and of the first balloon 500 of the two insertion instruments 300 and 400 are thus held attached to each other.

In addition, it will be noted that the two insertion instruments 300 and 400 are thus substantially positioned in line, and the diameter of the whole of the insertion system is reduced in the coronary artery.

The two attached insertion instruments 300 and 400 and the stents 100 and 200 that they are carrying are then inserted into the associated guide catheter.

The flexible guide 700, placed beforehand in the second branch B2 of the bifurcation, is then inserted by its proximal end in the free end 526 of the nose 523 of the first balloon 500 of the second insertion instrument 400.

The assembly that includes the two attached insertion instruments 300, 400 moves along this guide 700 through the guide catheter and then the main section P of the blood vessel V to be treated until it reaches the bifurcation.

Thus, the removable auxiliary tube 530/mandrel 800 assembly holding together the two noses 522, 321 of the first balloon 500 and of the main balloon 310 during their progression along the single guide 700 toward the bifurcation enables us to eliminate the risks of crossing the guides.

We stop the progression of the insertion system at the sheath of the bifurcation and we release the nose 523 of the first balloon 500 held by the rigid mandrel 800.

To this end, in a first step, we undo the torquer T2 of the connection system 325 of the inflation tube 322 from the main balloon 310, so that the mandrel 800 is then free to move.

In a second step, we withdraw this mandrel 800 from the auxiliary tube 530 freeing the two noses 523 and 321 from the two balloons 310 and 500.

We then replace it in the guiding channel 323 associated with the main balloon 100 by a flexible guide that is pushed into the first branch B1 of the bifurcation.

We then push on each of the two guides pre-positioned in the two branches B1 and B2 of the bifurcation.

The two balloons 500 and 310, freed from each other, and the associated insertion instruments 400 and 300 advance in their respective flexible guides and respectively separate in the two branches (B1 and B2) of the bifurcation.

Following this movement, firstly the distal portion 130 of the main stent 100 is located in the first branch 131, the stent 100 having advanced along its guide until the orifice 140 is positioned at the entrance of the second branch B2 of the bifurcation.

Secondly, the nose 523 and a first half of the first balloon 500 passing through the orifice 140 are inserted into the second branch B2 of the bifurcation, while the second half of the first balloon 500 and the rest of the second insertion instrument 400 placed in the proximal portion 120 of the main stent 100 are located in the main section P of the blood vessel V.

This precise position is marked by the correspondence of the respective radio-opaque markers 540 and 102 of the first balloon 500 and of the orifice 140 located at the sheath of the bifurcation.

The insertion system is now in place and ready to treat the lesions in the bifurcation.

b. Deployment of the System for the Fitting of Stents

Until now, the balloon assembly has remained in the deflated state.

Since the position of the orifice 140 and of the first balloon 500 at the sheath of the bifurcation has been ensured by virtue of the radio-opaque markers 102, 540, the balloons can now be inflated.

In order to deploy the system, in a first step, the first balloon 500 of the second insertion instrument 400 is inflated by means of its inflation tube 521.

As illustrated in FIG. 5c, this inflation is used firstly to dilate the mesh of the orifice 140 through which it is passing and to shape the latter to the entrance of the second branch B2 of the bifurcation. Secondly, it is also used to dilate the proximal portion 120 of the main stent 100. This balloon 500 is then deflated.

In a second step, as illustrated in FIG. 5d, the inflation of the main balloon 310 is used to fully deploy the associated main stent 100 which is well open.

The main balloon 310 is then deflated.

At this level, the mesh of the tubular element 101 of the main stent 100 covers the first branch B1 of the bifurcation and the main section P of the blood vessel, and is positioned facing the second branch with its orifice 140, with a dilated mesh traversed by the first balloon 500 of the second insertion instrument 400.

In a third step, knowing the distance separating the two colour markers R1 and R2, respectively carried by the common element 410 of the second insertion instrument 400 and by the inflation tube 322 of the first insertion instrument 300, we withdraw the main balloon 310 so that the colour marker R2 coincides with the colour marker R1 of the fixed common element 410, as illustrated in FIGS. 6a and 6b.

An angiographic test is then performed.

If the result of insertion the main stent 100 is satisfactory and optimal at the two branches B1 and B2 of the bifurcation, then it is not necessary to go on to the deployment of the secondary stent 200 in the second branch B2. We then quite simply withdraw the two insertion instruments 300, 400 and the procedure is finished.

If not, and if the result is not optimal in the second branch B2, we then move the second insertion instrument 400 so that the second balloon 600 carrying the secondary stent 200 is placed in the second branch B2 after passing through the orifice 140 of the main stent 100.

Advantageously, as illustrated in FIG. 5e, the linear arrangement of the two balloons 500, 600 of the second insertion instrument 400 facilitates the penetration of the mesh of the orifice 140 by the second balloon 600 in order to install the secondary stent 200.

Knowing the distance separating the two colour markers R3, R2 carried by the inflation tube 322 of the first insertion instrument 300, we effect this movement by pushing the second insertion instrument 400 so as to make the colour marker R1 correspond with marker R3, as illustrated in FIG. 6c.

The position of the second balloon 600 is also controlled by the correspondence of the two radio-opaque markers 102 and 230 placed respectively on the orifice 140 and on the bevel 202 of the main 100 and secondary 200 stents, as illustrated by FIGS. 5e and 5f.

After verifying that the second balloon 600 has been positioned correctly, it is then deflated in order to deploy the secondary stent 200.

This is shaped firstly to the wall of the second branch B2 and secondly, at its bevelled end, mates with the dilated orifice 140 of the main stent 100.

As illustrated in FIGS. 4 and 5f, the main 100 and secondary 200 stents are then deployed so as to cover the two branches B1 and B2 of the bifurcation and the main section P of the blood vessel V treated.

The treatment of the lesions at the bifurcation is thus complete.

We perform a final angiographic test in order to verify that the arrangement and the deployment of the two stents 100 and 200 are optimal.

If they are, we then withdraw the two insertion instruments 300 and 400 at the same time, and the treatment of the lesions in the blood vessel V bifurcations, using the insertion system of the invention, is finished.

If they are not, it is also possible to repeat dilation of the main balloon 310 and of the first balloon 500, together, using a known technique, known as the kissing-balloon technique, which will not be described here.

Those skilled in the art will be able to understand an angioplasty system for the treatment of lesions in blood vessel bifurcations that, in relation to the known devices of the prior art, has an insertion system that allows the insertion of a stent into a first branch B1 of the bifurcation and leaves the choice, to the operator, of whether to insert a second in the second branch B2 if need be, during the same procedure.

In addition, such an insertion system can be used in a simple, reliable and effective way without the risk of crossing the metal guides directing the two stents 100 and 200 in the respective branches B1 and B2 of the bifurcation.

Naturally the present invention is not limited to the particular methods of execution that have just been described, but also extends to any variant that conforms to its spirit. In particular, the present invention is not limited to the appended drawings. The specific references illustrated in the preceding paragraphs are non-limiting examples of the invention.

1. A system for treating lesions on a blood vessel bifurcation, which includes a first branch and a second branch, the system being, characterised in that it includes:
a first stent adapted to be positioned, in part, in the first branch of the bifurcation;
a second stent adapted to be placed in the second branch of the bifurcation and to be inserted into this second branch by passing through an orifice of the first stent once the latter has been dilated and,
an insertion instrument with a first longitudinal axis, the said insertion instrument including:
a first balloon adapted to be inserted into the second branch through the orifice of the first stent and to dilate the said orifice;
a second balloon, aligned with the first balloon along the first longitudinal axis, adapted to receive the second stent and to dilate it.

2. The system according to the preceding claim, characterised in that the first and second balloons include a first common guiding channel lying along the said first axis, the said first guiding channel being adapted to receive a first guiding element that is intended to direct the assembly of the two balloons in the second branch of the bifurcation.

3. The system according to the preceding claim, characterised in that the first and second balloons are each extended, at a proximal end by an inflation tube that is adapted to receive the first guiding element and to inflate the balloon, the said inflation tubes lying substantially parallel to the said first longitudinal axis, being fixed together over part of their length.

4. The system according to the preceding claim, characterised in that the inflation tubes are welded together over part of their length.

5. The system according to claim 1, characterised in that the first stent is associated with a second insertion instrument with a second longitudinal axis and that includes at least one main balloon adapted to carry and to dilate the first stent, the said main balloon being extended at a proximal end by an inflation tube adapted to receive a second guiding element and to inflate the said balloon.

6. The system according to the preceding claim, characterised in that the first insertion instrument is in possession of reception means adapted to receive, in part, the second guiding element, and the second insertion instrument is in possession of means for reversible attachment of the said second guiding element to the reception means so as to hold the two insertion instruments together during their progression along the first guiding element up to the bifurcation.

7. The system according to the preceding claim, characterised in that the reception means include a hollow auxiliary tube adapted to receive the second guiding element at a proximal end, and to be attached at a distal end to a so-called nose tube that includes the first guiding channel, the said nose extending the first balloon from its distal end parallel to the first axis.

8. The system according to claim 6, characterised in that the means for reversible attachment include a connection system with a torquer in association with the second guiding channel to fix the second guiding element to the reception means.

9. The system according to claim 5, characterised in that the second guiding element is a mandrel.

10. The system according to claim 7, characterised in that the auxiliary tube is a tube lying from the nose in an inclined direction in relation to the said first axis toward the first balloon.

11. The system according to claim 7, characterised in that the auxiliary tube includes a distal end with a bevel adapted to be fixed to the nose by adhesion or welding.

12. The system according to claim 1, characterised in that the orifice has a mesh adapted to receive the first balloon and to open wide without deforming during its expansion, and adapted to assume the shape of the second stent carried by the second balloon that traverses said orifice at entrance of the second branch of the bifurcation.

13. The system according to claim 1, characterised in that the second stent has a proximal end with at least one bevel.

14. The system according to claim 3, characterised in that the inflation tubes include colour markers.

15. The system according to claim 1, characterised in that each balloon and each stent has at least one marker radio-opaque marker.

16. A method for treating lesions on a blood vessel bifurcation, which includes a first branch and a second branch, the method including the following steps:
positioning of a first stent, in part, in the first branch of the bifurcation; and
positioning of a second stent in the second branch of the bifurcation, the second stent being inserted into this second branch by passing through an orifice of the first stent once the latter has been dilated;
in which the step for positioning of the second stent is made by means of an insertion instrument with a first longitudinal axis and that includes a first balloon and a second balloon aligned with the first balloon along the first longitudinal axis, and that includes the following substeps:
insertion of the first balloon in the second branch through the orifice of the first stent and dilation of the said orifice;
reception of the second stent on the second balloon and dilation of the said second stent.

17. A system for treating lesions on a blood vessel bifurcation, which includes a first branch and a second branch, the system being characterized in that it includes:
a first stent comprising a proximal portion and a distal portion, said stent being adapted to be positioned, in part, in the first branch of the bifurcation and comprising an orifice in a lateral wall;
a first insertion instrument with a first longitudinal axis, that includes a main balloon adapted to be introduced into the first branch and to carry the first stent and dilate it; and
a second insertion instrument with a second longitudinal axis, including a first balloon adapted on the one hand to be inserted into the second branch through the orifice of the first stent to be positioned in part in the second branch, and on the other hand to dilate the said orifice and the proximal part of the first stent.

18. The system according to claim 17, characterized in that it further comprises a second stent adapted to be placed in the second branch of the bifurcation and to be inserted into said second branch by passing through the orifice of the first stent once the latter has been dilated, and that the second insertion instrument further comprises a second balloon, aligned with the first balloon along the second longitudinal axis, said second balloon being adapted to receive the second stent and to dilate it.

19. The system according to claim 18, wherein the first and second balloons include a first common guiding channel lying along the said second axis, the said first guiding channel being adapted to receive a first guiding element that is intended to direct the assembly of the two balloons in the second branch of the bifurcation.

20. The system according to claim 19, wherein the first guiding element is deprived of connecting system with torquer, and in that it is engaged from the beginning into the orifice of the first stent.

21. The system according to claim 18, wherein the orifice has a mesh adapted to receive the first balloon and to open wide without deforming during its expansion, and adapted to assume the shape of the second stem carried by the second balloon that traverses said orifice at entrance of the second branch of the bifurcation.

22. The system according to claim 18, wherein the second stent has a proximal end with at least one bevel.

23. The system according to claim 19, characterized in that the first and second balloons are each extended, at a proximal end by an inflation tube that is adapted to receive the first guiding element and to inflate the balloon, the said inflation tubes lying substantially parallel to the said second longitudinal axis, being fixed together over part of their length.

24. The system according to claim 23, characterized in that the inflation tubes are welded together over part of their length.

25. The system according to claim 23, wherein the inflation tubes include color markers.

26. The system according to claim 15, wherein said main balloon is extended at a proximal end by an inflation tube that is adapted to receive a second guiding element and to inflate the balloon.

27. The system according to claim 26, wherein the inflation tubes include color markers.

28. The system according to claim 19, characterized in that the second insertion instrument is in possession of reception means adapted to receive, in part, the second guiding element, and the first insertion instrument is in possession of means for reversible attachment of the said second guiding element to the reception means so as to hold the two insertion instruments together during their progression along the first guiding element up to the bifurcation.

29. The system according to claim 28, characterized in that the reception means include a hollow auxiliary tube adapted to receive the second guiding element at a proximal end, and to be attached at a distal end to a so-called nose tube that includes the first guiding channel, the said nose extending the first balloon from its distal end parallel to the second axis.

30. The system according to claim 28, characterized in that the means for reversible attachment include a connection system with a torquer in association with the second guiding channel to fix the second guiding element to the reception means.

31. The system according to claim 26, characterized in that the second guiding element is a mandrel.

32. The system according to claim 29, characterized in that the auxiliary tube is a tube lying from the nose in an inclined direction in relation to the said second axis toward the first balloon.

33. The system according to claim 29, characterized in that the auxiliary tube includes a distal end with a bevel adapted to be fixed to the nose by adhesion or welding.

34. The system according to claim 15, characterized in that each balloon and each stent has at least one marker radio-opaque marker.

35. A method for treating lesions on a blood vessel bifurcation, which includes a first branch and a second branch, said method comprising the following steps:
positioning a first stent in part in the first branch of the bifurcation, said stent comprising a proximal portion, a distal portion, and an orifice in a wall;
introducing a first balloon into the second branch through the orifice of the first stent;
positioning said first balloon at least in part into the second branch;
inflating said first balloon in order to dilate the orifice and the proximal portion of the first stent.

36. A method for treating lesions on a blood vessel bifurcation, which includes a first branch and a second branch, said method comprising a step consisting of positioning a first stent in part in the first branch of the bifurcation, said stent comprising an orifice in a wall, wherein the positioning step of the first stent is implemented by using a first guiding element positioned in the second branch.

37. The system according to claim 26, characterized in that the second insertion instrument is in possession of reception means adapted to receive, in part, the second guiding element, and the first insertion instrument is in possession of means for reversible attachment of the said second guiding element to the reception means so as to hold the two insertion instruments together during their progression along the first guiding element up to the bifurcation.
